(19) United States
(12) Patent Application Publication
(10) Pub. No.: US 2023/0241358 A1
(43) Pub. Date: Aug. 3, 2023

(54) A PERFUSION BALLOON WITH AN EXPANDABLE INTERNAL LUMEN

(71) Applicant: Mehmet Hakan AKPINAR, Kadikoy/Istanbul (TR)

(72) Inventor: Mehmet Hakan AKPINAR, Kadikoy/Istanbul (TR)

(21) Appl. No.: 18/003,212

(22) PCT Filed: Jul. 1, 2021
(86) PCT No.: PCT/TR2021/050680

§ 371 Date: Dec. 23, 2022

Related U.S. Application Data
(60) Provisional application No. 63/047,306, filed on Jul. 2, 2020.

(51) Int. Cl. A61M 25/10; A61M 5/00; A61M 25/00; A61M 60/13; A61M 60/295; A61M 60/31; A61M 60/497; A61M 60/843

(52) U.S. Cl. CPC A61M 25/10; A61M 5/007; A61M 25/0097; A61M 60/13; A61M 60/295; A61M 60/31; A61M 60/497; A61M 60/843; A61M 2025/105; A61M 2210/1032; A61M 2210/12; A61M 2025/1079; A61M 2025/1095; A61M 2205/0238

(57) ABSTRACT

The present invention relates to a perfusion balloon catheter that has an expandable (enlargeable) inner lumen (114) with a novel design feature that allows a continuous flow through this enlarged lumen sufficient perfusion to the distal lumen of the blood vessel or air passage concurrent to and independently of balloon (104) inflation or deflation resulting in the prolonged dilatation and avoiding the high risks of blood or air flow stricture during balloon inflation.

### TECHNICAL FIELD OF THE INVENTION

The present invention relates to a perfusion balloon catheter that has an expandable (enlargeable) inner lumen with a novel design feature that allows a continuous flow through this enlarged lumen sufficient perfusion to the distal lumen of the blood vessel or air passage concurrent to and independently of balloon inflation or deflation resulting in the prolonged dilatation and avoiding the high risks of blood or air flow stricture during balloon inflation.

### BACKGROUND OF THE INVENTION (PRIOR ART)

Balloon dilation is performed to relieve stenosis of valves, vessel walls, surgically-created pathways, or intracardiac structures such as a fenestration in the atrial septum or restrictions of the airway. There have been significant advances in the size, profile, design, and materials used in balloon catheters, facilitating their use in various applications. Tracheobronchial balloon dilation and stent placement have been well used in the treatment of patients with benign and/or malignant diseases. Under direct vision using a FOB or rigid bronchoscope, a balloon catheter is threaded over a guidewire and positioned across the stenotic airway. The balloon is inflated for 30 to 120 seconds with repeat inflation-deflation sequences if airway narrowing persists. Although balloon dilation is simple and fast, the recurrence rate is high. Airway dimension increases with immediate relief of symptoms. However, the standard balloon catheters allow only a limited duration of inflation so balloon dilatations must be kept for a short time to avoid significant damage to the vital organs and patient discomfort. Therefore, the results are usually temporary, and many patients require serial dilatations, an airway stent, or laser therapy.

The balloon catheters are also used to open pulmonary arteries that have been narrowed or blocked artery and thus provides blood flow to the lungs, reduces shortness of breath, and improves exercise tolerance. More data on its safety and effectiveness are still required. The pulmonary arteries have thinner walls than the heart's blood vessels, so an injury by rupture or dissection resulting in hemorrhage is a risk.

Injury to the lung, including reperfusion edema, is also possible, and these injuries are less likely with more experienced specialists. The perfusion balloon catheters are currently used in many minimally invasive procedures to provide treatment of a blockage or narrowed vessels in many parts of the body or occlusion of a disrupted vessel to control hemorrhage.

Balloon pulmonary angioplasty (BPA) is one of the used areas of the perfusion balloon catheters. BPA is an emerging minimally invasive procedure to treat chronic thromboembolic pulmonary hypertension (CTEPH) in people who are not suitable for pulmonary thromboendarterectomy (PTE) or have residual pulmonary hypertension and areas of narrowing in the pulmonary arterial tree following the previous PTE. Another use area of the perfusion balloons is hemorrhage control. Hemorrhage is the leading cause of potentially preventable death after a traumatic injury. Noncompressible torso hemorrhage (NCTH) is defined as vascular disruption of the axial torso vessels, the pulmonary parenchyma, solid organs, or the bony pelvis. The key objective of resuscitation is to stop the bleeding and restore circulating blood volume to avoid mortality in these patients. Endovascular techniques to treat hemorrhage in the pelvis, spleen, liver, and kidney also continue to evolve. Resuscitative endovascular balloon occlusion of the aorta (REBOA) is the newest endovascular technique of achieving inflow control to temporarily slow NCTH. It involves placing a proper size balloon occlusion catheter into the aorta via the common femoral artery (CFA). The greatest limitation to REBOA is the ischemia caused by total aortic occlusion. An additional serious limitation of REBOA is the need for rapid and accurate placement. The issue of ischemia-reperfusion injury from complete occlusion of the aorta by REBOA has led to research into “partial-REBOA” or “P-REBOA” which allows titration of partial deflation of the balloon to allow some distal perfusion while maintaining afterload, possibly by an automated device. Studies have been limited to two animal reports.

Prolonged balloon inflation with a perfusion balloon catheter is ideal at low pressure to seal the perforation, maintaining myocardial perfusion. In the passive auto-perfusion balloon catheter with a dual lumen shaft, blood enters the lumen of the balloon catheter proximal to the balloon through side holes, travels along the balloon lumen then exits distally. In general, since the maximum perfusion rate through a catheter is determined primarily by the catheter's internal diameter, the prolonged dilatation for a continuous air or blood flow cannot be achievable with the standard perfusion balloon catheters. Another method that is used to provide hemostasis through the control of bleeding is the use of sealants. The sealant's disintegration process should not cause any unwanted or pathological process like immunological or other.

In the late 1980s, an “auto-perfusion catheter” device was described that allows blood to enter through proximal side holes, pass through a central lumen in the balloon, exit through distal, and provide prolonged balloon inflation. However, there is no data for an approved device regarding the described concept. ER-REBOA™ Catheter (Prytime Medical, Bourne, USA) is a balloon catheter to temporarily occlude large vessels and control hemorrhage through a minimally invasive technique. It has a guidewire-free design and an atraumatic catheter tip to prevent vessel damage. Reboa Kit (Reboa Medical, Norwegian) is the first approved kit to provide hemostasis in case of postpartum hemorrhage, a trauma in the abdomen or pelvis, bleeding during surgery, aortic rupture, prophylactic v. placenta percreta/accrete. The kit contains everything for the procedure that can pass without review and with the market's smallest available REBOA balloon catheters on 6 and 7 Fr.

The pericardium-covered stent (PCS) ‘Overand-Under™’ (IGTM Medical, Or Akiva, Israel) was the first in a series of heterologous tissue-covered stents designed to set a barrier between the stent and the vessel wall. It became commercially available in Europe for clinical use in 2006. It is designed to reduce complications by preventing distal embolization in a high-risk subset of patients.

The subsequent newer generations of this stent (i.e., the Aneugraft™ and the Aneugraft™ Dx [ITGI Medical, Or Akiva, Israel]), have been used for the urgent treatment of coronary perforations. The Aneugraft consists of a highly flexible, laser-cut, 316 L stainless-steel balloon-expandable stent covered with a single layer of the equine pericardium. The TRUE® FLOW Valvuloplasty Perfusion Catheter is engineered to be true to size, exhibiting less than 1% stretch between nominal and rated burst pressure (RBP). The TRUE® FLOW Valvuloplasty Perfusion Catheter is designed to provide low hemodynamic resistance on the balloon, while inflated BD's proprietary fiber-based shell is designed to be rupture resistant. Interventionists sometimes use long inflation during endovascular treatment, which helps overcome some deteriorated situations. However, long inflation could induce hypercoagulability due to blood congestion and ischemic symptoms. Metacross RX (TERUMO, Tokyo, Japan) was developed as the first 0.035″-compatible rapid-exchange balloon for peripheral use in 2016 to prevent this effect. This device enabled long inflation in the coronary artery to bailout vessel perforation and to get thrombus control. The INSPIRA AIR® Balloon Dilation System comprises of high pressure, non-compliant balloon catheter, and an integrated stylet. Optimized for airway anatomy, the INSPIRA AIR® Balloon Dilation System is engineered to deliver controlled radial dilation of airway strictures with atraumatic access. CRE Pulmonary Balloons (Boston Scientific, USA) are also used to dilate the strictures in the airway. It allows serial balloon dilations utilizing multiple balloons.

### SUMMARY OF THE INVENTION

The present invention relates to a device that provides a prolonged dilatation avoiding the high risks of blood or air flow structure during balloon inflation and manufacturing such a device.

An object of the present invention is to prevent damage to organs by ensuring air or blood flow in the diseased or damaged area, which is vital for the success of the medical procedure.

Another object of the present invention is to provide a perfusion balloon catheter that has an expandable inner lumen with a novel design feature that allows a continuous flow through this enlarged lumen sufficient perfusion to the distal lumen of the blood vessel or air passage concurrent to and independently of balloon inflation or deflation.

### BRIEF DESCRIPTION OF THE DRAWINGS

FIG. 1 is a schematic illustration of a preferred embodiment of the perfusion balloon catheter device.

FIG. 2 is a schematic illustration of a preferred embodiment of the perfusion balloon catheter device.

FIG. 3 is a schematic illustration of a preferred embodiment of the perfusion balloon catheter device.

FIG. 4 is a schematic illustration of a preferred embodiment of the perfusion balloon catheter device.

FIG. 5 is a schematic illustration of a preferred embodiment of the perfusion balloon catheter device.

FIG. 6 is a schematic illustration of a preferred embodiment of the perfusion balloon catheter device.

FIG. 7 is a schematic illustration of preferred embodiments of the perfusion balloon catheter device.

### DESCRIPTION OF REFERENCES IN FIGS

101) self/mechanically expandable stent frame
102) inner lumen
103) radiopaque marker band
104) balloon
105) kink-resistant inflation lumen
106) connecting strut
107) balloon catheter main shaft
108) fenestration holes
109) connecting part of catheter
110) internal leaflets
111) external sleeve
112) catheter hub
113) Y connector hub
114) internal lumen
115) external surface
116) internal part

### DETAILED DESCRIPTION OF THE INVENTION

The present invention relates to a perfusion balloon catheter device withan expandable inner lumen that allows a continuous flow through this enlarged lumen sufficient perfusion to the distal lumen of the blood vessel or air passage concurrent to and independently of balloon inflation or deflation.

The perfusion balloon catheter device has a balloon (104) with an expandable internal lumen (114) to permit sufficient blood flow during the balloon inflation time. Balloon dilatation time is limited because of blocking the distal perfusion.

The subject matter of the device creates a large perfusion lumen inside the balloon catheter at the inflated balloon's location. The subject matter of the device has an expandable internal balloon lumen with a self-expandable or mechanically expandable internal lumen ((114) at the balloon inflation zone of the catheter. The perfusion balloon catheter device has an internal surface or a metallic structure which either self or mechanically expands to create the large internal lumen (114) between the balloon surfaces to provide sufficient blood perfusion through the balloon catheter lumen afterward, the balloon is inflated to a nominal pressure to perform a thoracic intervention, angioplasty, vessel wall occlusion or drug-release for a certain period without blocking the blood flow and supplying sufficient perfusion for the distal tissues and vessels.

In an embodiment of the invention, the perfusion balloon catheter device comprises a balloon catheter main shaft (107) that provides all device components are connected to advance and navigate together, to push, pull and steer together. In addition, the perfusion balloon catheter device also comprises a kink-resistant inflation lumen (105) to inflate and deflate the balloon at the desired location, which is connected to the balloon catheter main shaft (107) and Y connector hub (113), which is inflation part of catheter hub (112).

One embodiment of the invention device includes an external sleeve (111) to advance catheter and balloon (104) to the desired location with a minimum possible profile. The catheter hub (112) of the external sleeve (111) is pulled back to expand self/mechanically expandable stent frame (101) of the internal lumen (114) of the balloon to create a large lumen for air or blood perfusion. Expanding the balloon (104) to a certain diameter to start inflation to achieve enough pressure provides performing a thoracic intervention, angioplasty, vessel wall sealing or drug-elution for a longer period than conventional systems.

The device is advanced through an introducer sheath and over a compatible guidewire to the desired location. The external sleeve (111) is pulled back to release the self/mechanically expandable stent frame (101) of the balloon catheter to enlarge the stricture for air or blood perfusion before the inflation of the balloon with an indeflator to a certain diameter and pressure. The invention device can stay inflated as desired without harming and distal tissued due to a lack of perfusion. The invention device can be deflated with the same indeflator and a vacuum can be applied to aspirate all the residual contrast media or air inside the balloon to push the external sleeve (111) and take all the system inside the external sleeve (111) to finalize the intervention and take all the system out of the patient.

The perfusion balloon catheter device comprises;
An internal lumen (114) made of Nylon-12, polyether block amide (PEBA), or polyurethane (PU) attached to the external surface of the self/mechanically expandable stent frame (101),
a self/mechanically expandable stent frame (101) made of a shape-memory metallic wire like nitinol,
an inner lumen (102) for the balloon catheter system used as a guidewire lumen made of Nylon-12, polyether block amide (PEBA), or polyurethane (PU),
a radiopaque marker band (103) made of platinum-iridium to visualize the balloon location under fluoroscopy,
a kink-resistant inflation lumen (105) for the balloon catheter made of Nylon-12, polyether block amide (PEBA), or polyurethane (PU);
a connecting strut (106) of the self/mechanically expandable stent frame (101) to the external surface of the inner lumen (102) made of shape-memory metallic wire like nitinol,
a balloon catheter main shaft (107) to push and pull the total system made of Nylon-12, polyether block amide (PEBA), or polyurethane (PU),
one or more fenestration holes (108) to create perfusion of the connection part of the catheter between two or more balloon parts of the system,
a connecting part of the catheter (109) between two or more balloon parts of the system
internal leaflets (110) of the balloon to use for pulsative perfusion needs to be created,
an external sleeve (111) to keep the self/mechanically expandable stent frame (101) in the crimped form until delivered to the desired location made of Nylon-12, polyether block amide (PEBA), or polyurethane (PU),
a catheter hub (112) of the external sleeve (111) to push and pull to expand and retrieve the self/mechanically expandable stent frame (101) part of the catheter,
a Y connector hub (113) of the balloon catheter system to be used as a guidewire lumen and the inflation and deflation of the balloon (104).

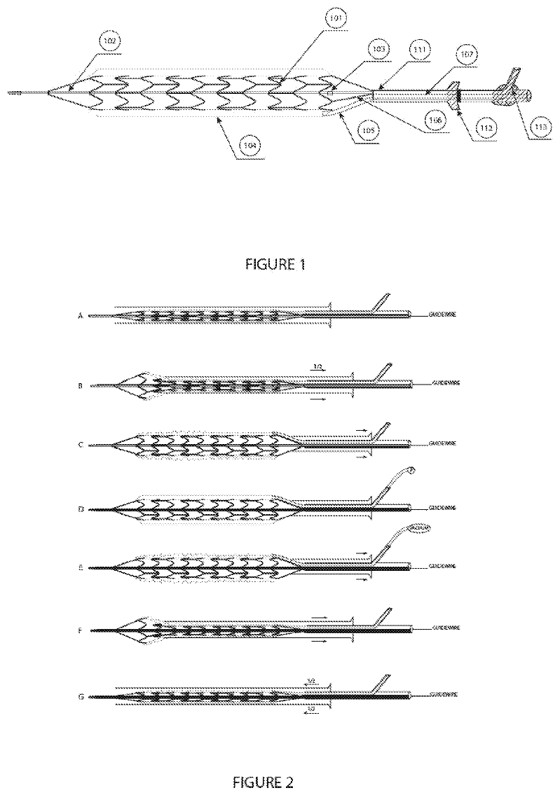

FIG. 1 is a schematic illustration of a preferred embodiment of the perfusion balloon catheter device of the invention having component details such as a self/mechanically expandable stent frame (101) as the skeleton of the enlarged internal lumen (114), an inner lumen (102) for the balloon catheter system used as a guidewire lumen, a radiopaque marker band (103) to visualize the balloon location under fluoroscopy, the balloon (104) part of the catheter attached to the external surface of the self/mechanically expandable stent frame (101), a kink-resistant inflation lumen (105) for the balloon catheter, the connecting strut (106) of the self/mechanically expandable stent frame (101) to the external surface of the inner lumen (102), the balloon catheter main shaft (107) to push and pull the total system, an external sleeve (111) to keep the self/mechanically expandable stent frame (101) in the crimped form until delivered to the desired location, a catheter hub (112) of the external sleeve (111) to push and pull to expand and retrieve the self/mechanically expandable stent frame (101) part of the catheter, a Y connector hub (113) of the balloon catheter system to be used as a guidewire lumen and the inflation and deflation of the balloon (104) part.

FIG. 2 is a schematic illustration of a preferred embodiment of the perfusion balloon catheter device of the invention having action mechanism details. FIG. 2A is a schematic illustration of a preferred embodiment of the perfusion balloon catheter device of the invention that shows the device inside of the delivery sheath. FIGS. 2B and 2C are schematic illustrations of a preferred embodiment of the perfusion balloon catheter device of the invention showing details of half and full releasing of the device, respectively. FIG. 2D is a schematic illustration of a preferred embodiment of the invention's perfusion balloon catheter device that describes the pressure applied to the balloon to inflate it. FIG. 2E is a schematic illustration of a preferred embodiment of the perfusion balloon catheter device of the invention that describes the vacuum application to deflate the balloon. FIGS. 2F and 2G are schematic illustrations of a preferred embodiment of the perfusion balloon catheter device of the invention that describe the half and full retraction of the device into the delivery system, respectively.

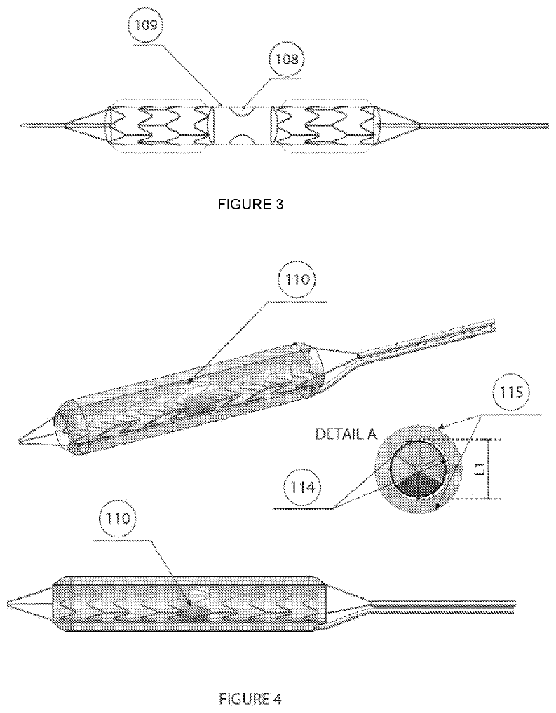

FIG. 3 is a schematic illustration of a preferred embodiment of the perfusion balloon catheter device of the invention showing details such as a fenestration hole (108) to create perfusion of the connection part of the catheter (109) between two or more balloon parts of the system and a connecting part of the catheter (109) between two or more balloon (104) parts of the system.

FIG. 4 is a schematic illustration of a preferred embodiment of the perfusion balloon catheter device of the invention showing details of the internal leaflets (110) of the balloon to use for pulsative perfusion that needs to be created. The internal leaflets (110) having an external surface (115) to seal blood or air flow between the vessel wall and the internal lumen (114),are attached to the internal surface of the self/mechanically expandable stent frame (101) and enlarged internal lumen (114) to function when the catheter is fully expanded.

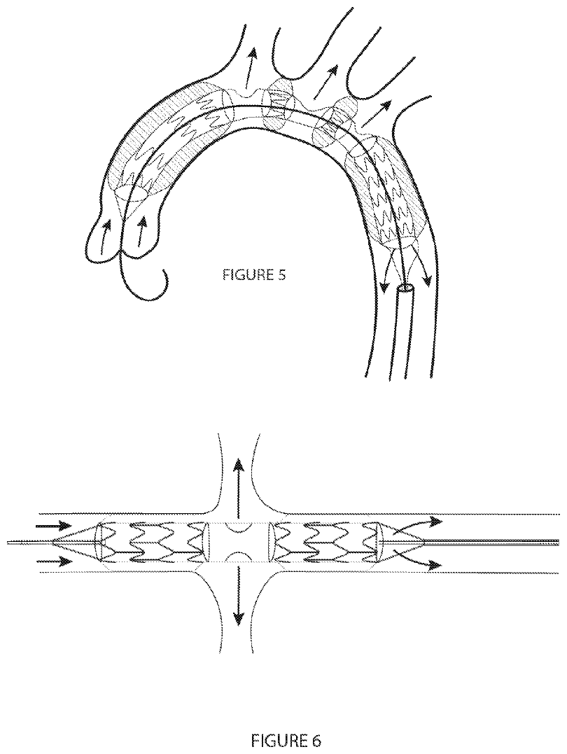

FIG. 5 is a schematic illustration of a preferred embodiment of the perfusion balloon catheter device of the invention that can be also utilized for one or more side branch(s) with different length and dimensions such as aortic arch, bifurcation, and trifurcation without blocking the side branches.,

FIG. 6 is a schematic illustration of a preferred embodiment of the perfusion balloon catheter device of the invention that can control the amount of the perfusion with the diameter of the fenestration holes (108) on the connecting part of the catheter (109) to regulate the flow.

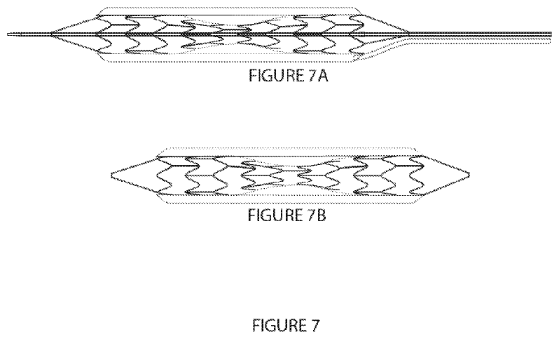

FIGS. 7A and 7B are schematic illustrations of a preferred embodiment of the perfusion balloon catheter device of the invention funneled to the center of the device, which creates increased flow resistance creating a back-pressure in the coronary arteries supporting enhanced microcirculation can be also utilized to regulate the blood flow.

1. A perfusion balloon catheter device which creates pulsative perfusion of air, blood or any liquid during balloon inflation in trachea, blood vessel, artery or vein with a constant flow, characterized by comprising;
An internal lumen (114) made of Nylon-12, polyether block amide (PEBA), or polyurethane (PU) attached to the external surface of the self/mechanically expandable stent frame (101),
a self/mechanically expandable stent frame (101) utilized as a skeleton to increase the cross-section of the blood perfusion lumen to create sufficient blood perfusion to the distal vessel or tissues which is made of a shape-memory metallic wire,
an inner lumen (102) for the balloon catheter system used as a guidewire lumen made of Nylon-12, polyether block amide (PEBA), or polyurethane (PU),
a radiopaque marker band (103) made of platinum-iridium to visualize the balloon location under fluoroscopy,
a kink-resistant inflation lumen (105) for the balloon catheter made of Nylon-12, polyether block amide (PEBA), or polyurethane (PU),
a connecting strut (106) of the self/mechanically expandable stent frame (101) to the external surface of the inner lumen (102) made of shape-memory metallic wire,
a balloon catheter main shaft (107) to push and pull the total system made of Nylon-12, polyether block amide (PEBA), or polyurethane (PU),
one or more fenestration holes (108) to create perfusion of the connection part of the catheter between two or more balloon parts of the system,
a connecting part of the catheter (109) between two or more balloon parts of the system
internal leaflets (110) of the balloon to use for pulsative perfusion needs to be created,
an external sleeve (111) to keep the self/mechanically expandable stent frame (101) in the crimped form until delivered to the desired location made of Nylon-12, polyether block amide (PEBA), or polyurethane (PU),
a catheter hub (112) of the external sleeve (111) to push and pull to expand and retrieve the self/mechanically expandable stent frame (101) part of the catheter,
a Y connector hub (113) of the balloon catheter system to be used as a guidewire lumen and the inflation and deflation of the balloon (104).

2. A perfusion balloon catheter device according to claim 1; wherein the self/mechanically expandable stent frame (101) has an enlargement structure to expand the internal lumen (114), a compliant, semi-compliant and non-compliant balloon (104) embedded over the self/mechanically expandable stent frame (101) and inflation lumen and an inner lumen (102) as a guidewire lumen and an external sleeve (111) to expand and crimp the self-expandable structure.

3. A perfusion balloon catheter device according to claim 2; wherein the device is constructed over the balloon catheter main shaft (107) and placed in an external sleeve (111) to create a smaller French calibration catheter and large size internal lumen (114) for the system to have sufficient perfusion of the air or blood through the catheter system.

4. Use of a perfusion balloon catheter device according to any one of claims 1 to 3 for drug delivery from the surface of the balloon as coated with or without polymer-based drug coating to create a release kinetic without any time limitation.

5. Use of a perfusion balloon catheter device according to any one of claims 1 to 3 for drug release from both sides of the balloon (104) surface internally and externally to release different drugs for different purposes.

6. Use of a perfusion balloon catheter device according to any one of claims 1 to 3 for drug release, wherein drug elution from the external surface can be programmed differently from the internal surface of the balloon (104).

7. Use of a perfusion balloon catheter device according to any one of claims 1 to 3 for drug release, wherein the guidewire lumen can be utilized to inject drug, contrast media.

8. A perfusion balloon catheter device according to any one of claims 1 to 3; wherein the internal leaflets (110) are located inside the balloon lumen and attached to the internal frame of the self/mechanically expandable stent frame (101) to create enough pressure gradient to transfer the amount of perfusion.

9. A perfusion balloon catheter device according to claim 8; wherein the resistance on the internal leaflets (110) can be adjusted to create the gradient difference to control the flow.

10. A perfusion balloon catheter device according to claim 8 or 9; wherein the internal leaflets (110) can also be activated from an external source of energy to increase and decrease the flow.

11. A perfusion balloon catheter device according to any one of claims 8 to 10; wherein the internal leaflets (110) can be designed as a rotodynamic pump to build up the pressure to increase the flow to create sufficient perfusion.

12. A perfusion balloon catheter device according to claim 1; wherein the connecting part of the catheter (109) between inflated balloons has one or more fenestration holes (108) to create sufficient air or blood perfusion or liquid transfer such as contrast media or drugs.

13. A perfusion balloon catheter device according to claim 1; wherein the device of the invention can be utilized for one or more side branch(s) with different lengths and dimensions such as the aortic arch.

14. A perfusion balloon catheter device according to claim 1; wherein the device has a structure which can create perfusion to bifurcation or trifurcation multivessel interventions without blocking the side branches.

15. A perfusion balloon catheter device according to claim 14; wherein the side branch fenestration holes (108) can be calibrated to determine the amount of the perfusion needed during the intervention.

16. Use of a perfusion balloon catheter device according to claim 1 for control the amount of the perfusion with the diameter of the fenestration holes (108) on the connecting part of catheter (109) to regulate the flow.

17. Use of a perfusion balloon catheter device according to claim 14 for complex interventions such as thoracic interventions, aortic arcus, renal arteries, 3A interventions, aneurysms, dissections, perforations or stenoses dilatation and keeping the sufficient air or blood perfusion to the distal vessel or tissues.

18. A perfusion balloon catheter device according to claim 14; wherein the connecting part of catheter (109) can have special material coating or surface treatment having anti-coagulation properties to avoid blood coagulation inside and outside of the system.

19. A perfusion balloon catheter device according to claim 14; wherein the device is funneled to the center of the device which creates increased flow resistance creating a back-pressure in the coronary arteries supporting enhanced microcirculation can be also utilized to regulate the blood flow.

20. A perfusion balloon catheter device according to claim 14; wherein the device can be detached from the main shaft and released at the desired location as a flow and pressure regulator in the coronary sinus.

21. Use of a perfusion balloon catheter device according to claim 14 for diagnostic purposes to change the calibration and the cross-section of the funneled part to measure the pressure changes and gradient.

22. A perfusion balloon catheter device according to claim 1, wherein self/mechanically expandable stent frame (101) is made of nitinol.

23. A perfusion balloon catheter device according to claim 1, wherein the inner lumen (102) is made of nitinol.
